(19) United States
(12) Patent Application Publication
(10) Pub. No.: US 2020/0000528 A1
(43) Pub. Date: Jan. 2, 2020

(54) MULTI-CAMERA IMAGING AND VISUALIZATION SYSTEM FOR MINIMALLY INVASIVE SURGERY

(71) Applicant: CHINMAY DEODHAR, PUNE (IN)

(72) Inventor: CHINMAY DEODHAR, PUNE (IN)

(21) Appl. No.: 16/487,851

(22) PCT Filed: Feb. 22, 2018
(86) PCT No.: PCT/IN2018/050092

§ 371 Date: Aug. 22, 2019

(30) Foreign Application Priority Data
Feb. 23, 2017 (IN) 201721006382

(51) Int. Cl. A61B 34/20; A61B 90/00; A61B 1/313; A61B 1/12; G06F 3/01

(52) U.S. Cl. CPC A61B 34/20; A61B 90/37; A61B 1/3132; A61B 2034/2065; G06F 3/017; A61B 2090/371; A61B 2034/2055; A61B 1/126

(57) ABSTRACT

Disclosed is a multi-camera imaging and visualization system for use in minimally invasive surgery. The system comprises a platform housing a computing system, a 5 frame configured on the platform, a shaft movably secured to lower end of the frame and a multi-camera system secured to the shaft. Specifically, the multi-camera system comprises a plurality of high resolution digital cameras housed in a frame shaped as a three-fourth circular ring, whereby the geometry of the camera system has a relatively smaller cross section area and is capable of being inserted through a small incision in 10 the abdominal wall, and yet provides a larger surface inside the abdomen to spread out the camera positions. The individual video feeds from each camera of the multi-camera system are rendered by the computer system and displayed to the surgeon.

### FIELD OF THE INVENTION

The present invention relates to a field of surgery. More specifically, the present invention relates to a multi-camera imaging, visualization and interaction system for minimally invasive surgery, by projecting images of internal organs, tissues, and surgical tools externally on a screen and being able to track hand gestures and tool movements by means of a camera viewing the screen.

### BACKGROUND OF THE INVENTION

Minimally invasive surgery (MIS) utilizes small incisions in the body for the placement and manipulation of surgical equipment. MIS has been widely adopted over the past few decades and performed as an alternative to open surgery because it minimizes trauma, shortens hospitalizations, and decreases recovery time.

While MIS provides many benefits, it often takes longer to complete than equivalent open surgeries. In particular, MIS is hindered by limited views and insertion points as the cameras used for visualization can only be inserted typically through up to 4 or 5 trocar incisions at specific positions. The field of view of the camera is limited by the orientation of the laparoscope, controlled by the surgeon or the assistant surgeon. Further, in order to gain a detailed view, the laparoscope is often placed very close to the area of interest, as a result creating clutter with other instruments, as well as preventing the surgeon from viewing the peripheral areas quickly. In order to view the peripheral areas, the laparoscope has to be physically moved around multiple times, which can cause further hindrances for the surgeon's movement of other tools as well. As a result of these and other limitations, MIS requires significantly more training than regular open surgery, which places an additional burden on the healthcare system, especially in remote and developing regions or low resource settings.

In order to view specific locations of interest during surgery as well as to gain a peripheral view of surrounding areas in the body, the laparoscope must be physically moved around by the surgeon and pointed to various locations. This causes interruptions in the surgical flow, as the surgeon must let go of instruments in one of their hands to operate the position and orientation of the laparoscope.

Often this task is carried out by an assistant surgeon. In such a situation, a surgeon must communicate to the assistant surgeon the correct orientation in which the laparoscope is desired. This creates several obstacles for smooth and efficient functioning of the surgery as there are often instances of miscommunication or under communication between what the surgeon wants to view and what the assistant surgeon points the laparoscope.

To overcome the above mentioned issues of visualization in minimally invasive surgery, many camera systems have been developed and used conventionally. Specifically, cameras are inserted into site of surgery which may include cameras on the front, for front and side viewing.

Further, robotic camera movement systems have been developed for use in MIS, but they all use complicated motor-drives, sensors, can be slow and expensive. The methods used for enabling the surgeon to provide commands to control the position and orientation of the camera are also not intuitive and user friendly.

Accordingly, there exists a need to provide a multi-camera imaging system for minimally invasive surgery which overcomes abovementioned drawbacks.

### OBJECTS OF THE INVENTION

An object of the present invention is to facilitate MIS by providing clear view of the operating site.

Another object of the present invention is to provide single camera unit for MIS which provides a complete peripheral view of the operating site.

Yet another object of the present inventions to provide for an imaging and visualization system that can effectively visualize various areas of the surgical site based on the needs of the surgeon and enabling the surgeon to interact with the system in a user friendly manner.

Yet another object of the present invention is to provide for a visualization system that can be utilized for image data storage, processing and implementation of artificial intelligence modules on surgical site video data to eventually help train and guide surgeons for future surgeries, based on data collected from past surgeries.

### SUMMARY OF THE INVENTION

Accordingly, the present invention provides a multi-camera imaging and visualization system for use in minimally invasive surgery. The system comprises a platform housing a computing system, a frame configured on the platform, a shaft movably secured to lower end of the frame and a multi-camera system secured to the shaft. Specifically, the multi-camera system comprises a plurality of high resolution digital cameras housed in a frame shaped as a three-fourth circular ring, whereby the geometry of the camera system has a relatively smaller cross section area and is capable of being inserted through a small incision in the abdominal wall, and yet provides a larger surface inside the abdomen to spread out the camera positions. The individual video feeds from each camera of the multi-camera system are rendered by the computer system and displayed to the surgeon.

The system further comprises a projector mounted on upper portion of the frame to display the images captured by the multi-camera system, a screen configured at lower portion of the frame adjacent to the multi-camera system for displaying the images, and at least one camera hoisted next to the projector to view the image created by the projector, wherein the camera tracks movements of tools and additional hand-gestures by a surgeon for manipulating the view.

### BRIEF DESCRIPTION OF THE DRAWINGS

FIGS. 1 and 2 show schematic representations of a multi-camera imaging and visualization system for minimally invasive surgery, in accordance with the present invention;

FIGS. 3-13 show various components of the multi-camera imaging and visualization system of FIG. 1;

FIG. 14 shows a plurality of multi-camera imaging systems communicating with a server for storing data; and

FIGS. 15 and 16 shows detailed view of the multi-camera system, in accordance with the present invention. In particular, FIG. 16 shows a typical cross sectional element at any given camera location (027)

### DETAILED DESCRIPTION OF THE INVENTION

The foregoing objects of the invention are accomplished and the problems and shortcomings associated with the prior art techniques and approaches are overcome by the present invention as described below in the preferred embodiment.

The present invention provides a multi-camera imaging and visualization system for use in minimally invasive surgery. The multi-camera imaging system records the images captured by a plurality of cameras attached on a frame and stitches together these images into one continuous video canvas of the entire surgical space. This arrangement allows surgeons to view any one specific location in the surgical site simply by viewing the digitally “trimmed” portion of the full video canvas but without having to physically move the camera system or any other device. Thus, the functions of a “robotic” camera can be achieved by this system, but without the use of any moving parts, thus making it more robust, cost effective and highly responsive.

The present invention also allows for visualization of the image captured by the camera system to be displayed on a screen, formed by a digital projector situated overhead. The location of the screen right next to the surgical site also allows for a more ergonomic visualization, which is much closer to what surgeons are familiar with in the open surgical environment, as opposed to the laparoscopy methods, where a screen is typically attached overhead.

The present invention is illustrated with reference to the accompanying drawings, throughout which reference numbers indicate corresponding parts in the various figures. These reference numbers are shown in brackets in the following description.

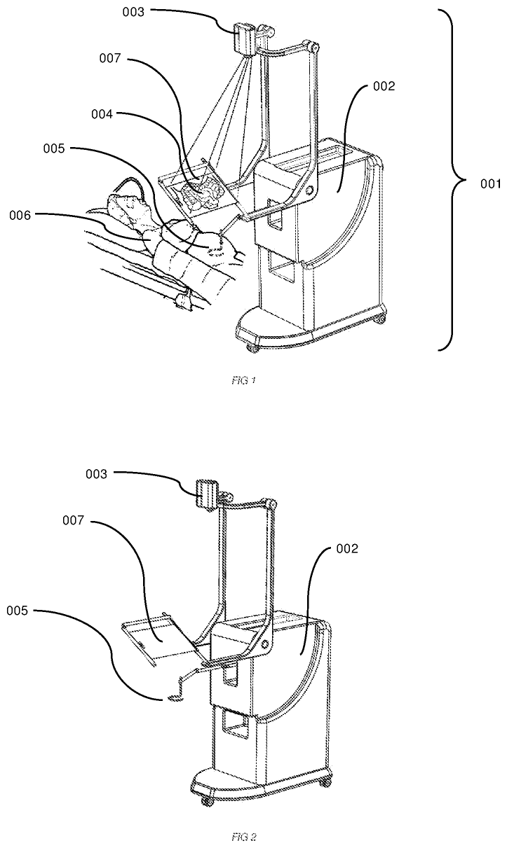

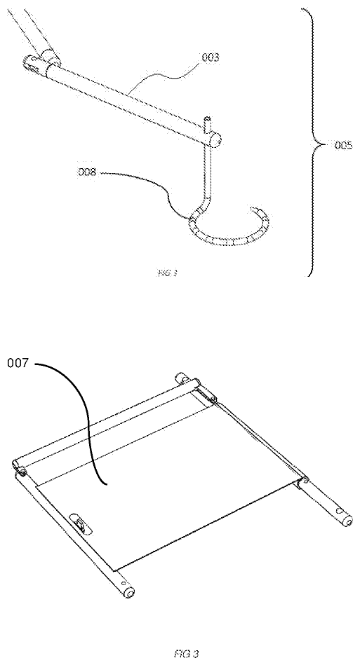

Referring now to FIGS. 1 and 2, there is shown a multi-camera imaging and visualization system (001) (hereinafter, the system (001)) in accordance with the present invention. The system (001) comprises a platform (002). In an embodiment, the platform (002) houses a computing system (not shown).

The computing system in the platform (002) further includes data storage capability, image processing capability and internet connectivity. The image processing capability is equipped with ability to analyze the video feed generated by the system. The analysis, based on the data gathered from past surgeries includes the abilities of:
1. Recognition of various organs in the body by means of image matching (similar to face recognition software widely available and used today). This is implemented by comparison of the overall shape and colour of various segments of a particular image with a previously recorded and stored database of known correlation between images and organs.
2. Recognition of the particular step that is currently ongoing at any given time, amongst all the sequential steps of that surgery. This is implemented by comparing the current imagery with the known states from previous surgeries from the database.
3. Ability to identify previous examples of similar situations if the surgeon wishes to view that. This is implemented by knowing the exact step in which the current surgery is ongoing and then being able to locate in the database further examples of that same moment in time showing the same step in similar previous surgeries as comparative examples to the surgeon.

These and similar abilities of the system act as a guide to the surgeon while performing surgeries, thus enabling less experienced surgeons to perform more complicated procedures.

The platform (002) comprises a frame configured thereon. The frame comprises a multi-camera system (005) adapted at one end of the lower portion thereof. The multi-camera system (005) is configured on an elongated shaft (003) which is capable of moving in all directions and capable of being extended as well as locked in a position set by the surgeon. In an embodiment, the shaft (003) is a telescopic shaft.

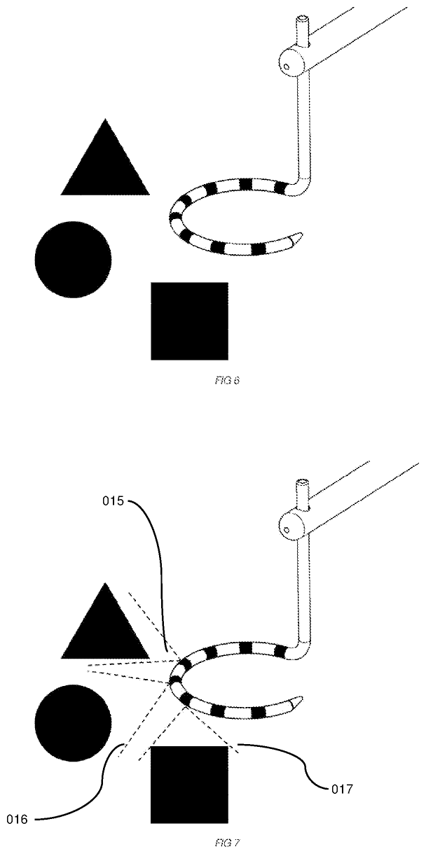

The multi-camera system (005) comprises a plurality of high resolution digital cameras (008) that point to all directions covering a complete internal view of the body as shown in FIG. 3. In a preferred embodiment of the present invention, the high resolution digital cameras (008) are housed in a frame shaped as a three-fourth circular ring as shown in FIG. 5. For the purposes of illustrating this invention, only three of the cameras are marked as (009), (010), and (011), although the system actually comprises a plurality of cameras, around 8 to 12 in number. In an embodiment, the geometry of the multi-camera system (005) has a relatively smaller cross section area and is capable of being inserted through a small incision in the abdominal wall, and yet provides a larger surface inside the abdomen to spread out the camera positions. Specifically, the frame with high resolution digital cameras (008) are inserted inside the abdomen through a single incision near the navel a shown in FIG. 6 around the organs to be imaged. The organs are generally represented by the numbers (012), (013) and (014). The digital images are transmitted to the computing system where they are integrated.

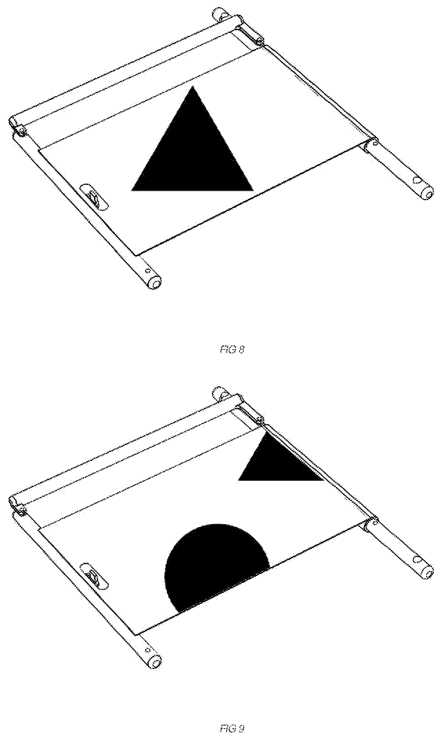

FIGS. 7 and 8 shows the multi-imaging camera system frame (005) surrounding internal organs. The digital cameras (008) are positioned within the three-fourth ring such that images are taken in all directions inside the abdominal cavity to provide a complete internal view of all the areas that the surgeon may be interested in. For the purposes of illustrating this invention, the fields of view of the three cameras (009), (010) and (011) are shown by dotted lines marked by (015), (016),and (017) respectively. In reality, the plurality of cameras would cover the entire internal area, thus enabling the surgeon to post-operatively view parts of the body that were away from the core surgical site. Additionally, the surgeon or an assistant may monitor the surrounding areas even during the surgery either on a separate display, or as an inset window within the main display. This is a significant advantage over the current state of the art as in the present situation, surgeons can focus only at a single particular location at a time and are unable to monitor other potential areas which can be risky.

In an embodiment, in the multi-camera system frame (005), the location of the cameras in the three-fourth circular ring or a partial circular ring is always fixed with respect to the navel of the patient. Further, this location is also constant for all patients across all hospitals and all surgeries. Hence, the data captured by the multi-camera system (005) becomes cross-compatible, thus significantly improving the learning ability of the artificial intelligence modules of the surgical system as well as of the master system module in the central server.

In another embodiment, the housing of the multi-camera system frame (005) is strong enough such that it can also be used as an abdominal wall lifting device. This can enable surgeons using this device to perform gasless surgeries, where the entire abdomen lifting force is supplied by the multi-camera system frame. Alternatively, in some cases, surgeons may perform low-pressure surgeries where part of the force to lift the abdomen is provided by the multi-camera system frame, while the remaining force is provided by a lower pressure of the gas, which typically is carbon-dioxide.

In another embodiment, the housing of the multi-camera system (005) further includes an irrigation channel (024) and suction channel (025), with nozzles pointed to the camera lenses (026), whereby the lens surfaces can be cleaned without having to remove the device out of the patient's body.

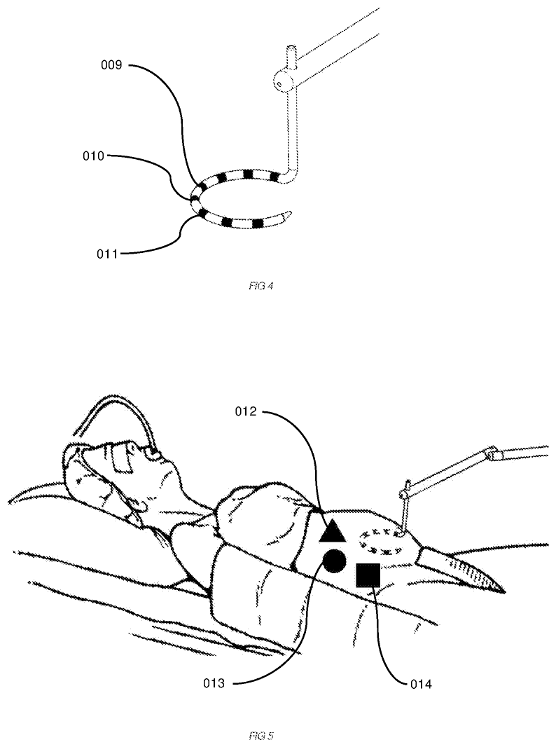

The platform (002) further comprises a projector (003) mounted on the upper portion thereof to display the image captured by the multi-camera system (005). The platform (002) furthermore comprises a screen (007) configured at the lower portion adjacent to the multi-camera system (005). The screen (007) as shown in FIG. 4 is capable of being adjustably positioned above the patient, at a location that is convenient and ergonomic for the surgeon and is able to be locked into such a position.

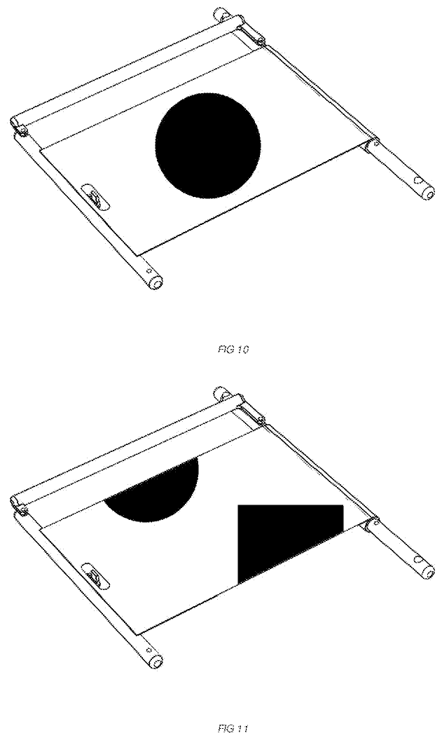

Specifically, individual video feeds from each camera, for example the camera (009, 010, 011) of the multi-camera system (005) are stitched into one continuous video canvas rendered by the computer system and displayed on the screen (007) through the projector (003). The surgeon can choose to view any portion of this video canvas which is of interest to them, with the typical functions of zoom, pan and rotate easily available as shown in FIGS. 9, 10, 11 and 12. For the purposes of illustrating this invention, the three cameras (090), (010) and (011) are positioned such that their fields of view (015), (016) and (017) show representative organs (012), (013) and (014) respectively.

For example, FIG. 9 shows the projection of the view (015) formed by the camera (009) and displaying the organ (012). In an ordinary situation, if the surgeon wanted to view the organ next to it (013), the complete laparoscope would have to be physically moved in that direction, either robotically or via the help of an assistant surgeon. In the present invention, this view would be form simply by digital manipulation of the continuous video canvas.

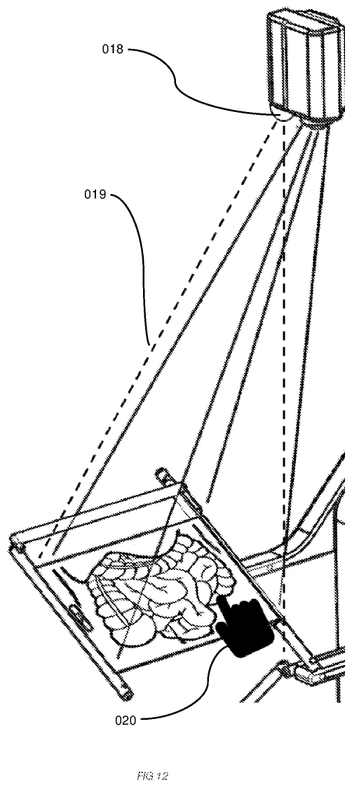

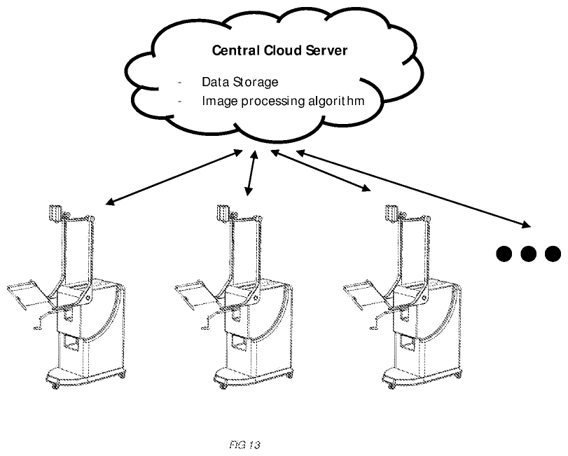

As shown in FIG. 10, a partial portion of the view (015) and view (016) are digitally trimmed and stitched together, to display partial positions of organs (012) and (013). Similarly, FIG. 11 shows view (016) of the camera (013) completely. FIG. 12 also illustrates a trimmed and re-stitched image of views (016) and (017).

These above described steps illustrate how the view can smoothly transition from viewing organ (012) to organ (013), resulting in an impression to the surgeon as if the camera moved from a direction oriented towards organ (012) to a direction oriented towards organ (013). In reality, however, there is no moving part involved in the current invention, as this smooth transition can occur merely by the digital manipulation of the images.

The system (001)further comprises at least one camera (018)mounted next to the projector (003) that can view the image created by the projector (003) as shown in FIG. 13. The image captured by the camera is generally represented by the number (019) in FIG. 13. The camera (018) is used for tracking movements of the tools and additional hand-gestures by the surgeon for manipulating the view and providing various customizable commands to the system. By knowing the image that is projected on the screen (007), and subtracting the image that is captured (019) by the camera (018), the position and shape of the any additional objects (such as a surgeon's hand (020) for capturing hand gestures) is easily calculated.

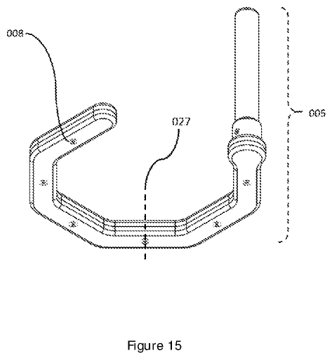

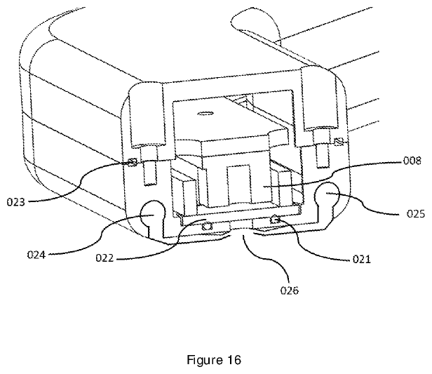

As shown in FIGS. 15 and 16, the multi-camera system (005) comprises lens (022) configured in front of the digital cameras (008). In another embodiment, the housing of the multi-camera system (005) is fitted with a lens cleaning device. In an embodiment, the lens can be easily cleaned by spraying warm water or other means either during or after surgery via the irrigation (024) and suction channels (025) running along the length of the device, and nozzles pointed directly at the exterior surface of the lenses (026).

In a preferred embodiment, the multi-camera system (005) further comprises an O-ring (021) and a silicone rubber gasket (023) that provides ingress protection, The O-ring (021) and a silicone rubber gasket (023) completely seal the electronic components inside a rigid enclosure. This allows the system to be sterilized and inserted into the patient's body for the duration of the surgery

The computer system records and stores the video captured by all cameras of the multi- camera system (005), while showing the surgeon only the portion that is of interest to him or her for the surgery. After the surgery, the video captured by the rest of the cameras may be viewed.

The computing system comprises a memory unit, processor and a module for tracking the movement of surgical instruments to display the relevant part of the view of the anatomy by following the position of the instruments. The module for tracking surgical instruments further comprises a predictive logic module to guide the system to display that portion of the canvas that is relevant for the next upcoming steps in the surgery.

The computing system furthermore comprises an artificial intelligence module that monitors the relative movements of the surgeon's instruments for specific procedures, thus learning to better predict the future anticipated movements of the instruments and associated visual field required for the subsequent steps of the surgery, thus contributing to the improvement of the module.

Further, video and image processing is carried out on the fully captured data from all the cameras of the multi-camera system (005). The analysis resulting from this processing gets stored in the computer system's local storage and is analyzed by artificial intelligence modules, which “learn” from more surgeries as they occur and contribute to the improvement of the machine learning. The key learning factors from this analyzed data is stored at a central server which can collect data from all surgeries across all hospitals w her e this system is deployed. In some embodiments, the raw image data captured by individual systems is also transmitted, stored and analyzed by the central server. This central server acts as a cloud system to collect and disseminate information to all individual systems deployed in various hospitals as shown in FIG. 14.

The entire system (001) may be used for improving the surgical outcomes by providing surgeons with an automated camera control system.

### Advantages of the invention

1. The system (001) shows surgeon only the portion that is of interest to him or her for the surgery, as well as provides an easy method to view the surrounding areas quickly and without movement of physical components, but my merely the digital manipulation of the data captured by multiple cameras. The video data from the surrounding areas is always captured and analyzed, however, and can also be monitored by the surgeon or an assistant even during the ongoing surgery. This reduces the risks associated with complications in the surrounding areas by enabling better monitoring of all surgical areas inside the body, and not just the surgical site which is being operated upon at any given moment.

2. The system (001) comprises a screen (007) which can be adjustably positioned above the patient, at a location that is convenient and ergonomic for the surgeon.

3. The system (001) allow surgeons to view any one specific portion of abdomen simply by viewing the digitally “trimmed” portion of the full video canvas but without having to physically move the camera system. This provides for a highly responsive visualization system, where the surgeon nearly instantaneously can view different parts of the surgical site with rapid succession.

4. The system (001) does not use any moving parts, thus making it more robust and cost effective.

5. The system (001) allows the surgeon to interact with use of hand gestures and tool tracking functions. The hand gestures enable the surgeon to interact with the system, provide the system with commands and manipulate the view by use of pan, zoom and rotate functions.

6. The projector based system allows implementation of a sterile system deployable in the surgical field, which is typically difficult with actual display monitors.

7. The video footage stored by the system (001) is a continuous canvas of the entire surgical area, captured from a relatively fixed position within the anatomy of the patient. This allows significantly improved image analysis to be performed by means of artificial intelligence modules that would be necessary for the future applications of surgical guides and tools. This cross-compatibility of the data significantly improves the learning capability of the artificial intelligence modules for image processing.

The foregoing objects of the invention are accomplished and the problems and shortcomings associated with prior art techniques and approaches are overcome by the present invention described in the present embodiment. Detailed descriptions of the preferred embodiment are provided herein; however, it is to be understood that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure, or matter. The embodiments of the invention as described above and the methods disclosed herein will suggest further modification and alterations to those skilled in the art. Such further modifications and alterations may be made without departing from the spirit and scope of the invention.

I claim:

1. A multi-camera imaging and visualization system for use in minimally invasive surgery comprising:
a platform housing a computing system;
a frame configured on the platform;
a shaft movably secured to lower end of the frame; and
a multi-camera system secured to the shaft, the multi-camera system comprising a plurality of high resolution digital cameras housed in a frame shaped as a three-fourth circular ring, whereby the geometry of the camera system has a relatively smaller cross section area and is capable of being inserted through a small incision in the abdominal wall, and yet provides a larger surface inside the abdomen to spread out the camera positions,
wherein individual video feeds from each camera of the multi-camera system are rendered by the computer system and displayed to the surgeon.

2. The system as claimed in claim 1, wherein the computing system comprises a memory unit, processor and a module for tracking the movement of surgical instruments to display the relevant part of the view of the anatomy by following the position of the instruments.

3. The system as claimed in claim 2, wherein the module further comprises a predictive logic module to guide the system to display that portion of the canvas that is relevant for the next upcoming steps in the surgery.

4. The system as claimed in claim 3, wherein the computing system further comprises an artificial intelligence module that monitors the relative movements of the surgeon's instruments for specific procedures, thus learning to better predict the future anticipated movements of the instruments and associated visual field required for the subsequent steps of the surgery, thus contributing to the improvement of the module.

5. The system as claimed in claim 1, wherein the housing of the multi-camera system is fitted with a lens cleaning device.

6. The system as claimed in claim 1, wherein the shaft is a telescopic shaft capable of being locked in a predefined position.

7. The system as claimed in claim 1, further comprising a projector mounted on upper portion of the frame to display the images captured by the multi-camera system.

8. The system as claimed in claim 7, further comprising a screen configured at lower portion of the frame adjacent to the multi-camera system for displaying the images.

9. The system as claimed in claim 8, wherein the screen is capable of being adjustably positioned and locked into a desired position above the patient's abdomen, whereby the line of sight of the surgeon to view the displayed images, is nearby the actual organs.

10. The system as claimed in claim 8, further comprising at least one camera hoisted next to the projector to view the image created by the projector, wherein the camera tracks movements of tools and additional hand-gestures by a surgeon for manipulating the view.
